(19) United States
(12) Patent Application Publication
(10) Pub. No.: US 2008/0243091 A1
(43) Pub. Date: Oct. 2, 2008

(54) Process and System For Systematic Oxygenation and Renal Preservation During Retrograde Perfusion of the Ischemic Kidney

(75) Inventors: Mitchell R. Humphreys, Scottsdale, AZ (US); Mark H. Ereth, Rochester, MN (US); Matthew T. Gettman, Rochester, MN (US)

Correspondence Address: BANNER & WITCOFF, LTD., TEN SOUTH WACKER DRIVE, SUITE 3000, CHICAGO, IL, 60606, US

(73) Assignee: Mitchell R. Humphreys, Scottsdale, AZ (US)

(21) Appl. No.: 11/843,339

(22) Filed: Aug. 22, 2007

Related U.S. Application Data
(60) Provisional application No. 60/839,367, filed on Aug. 22, 2006.

(51) Int. Cl. A61M 37/00

(52) U.S. Cl. 604/264

(57) ABSTRACT

A delivery system to provide end organ oxygenation and even systematic oxygenation in the face of ischemic result. The deliver system including a retrograde oxygenation and perfusion stent. The stent employing at least two and possibly more channels to allow flow of the perfusate from the device to the renal pelvis then to a back out to a collection apparatus. The stent may include various vital sign monitors, such as a renal pressure monitor, temperature monitor, and even an oxygenation monitor. The stent may include an anchoring device to allow the stent to be anchored into the renal pelvis in a temporary way during the retrograde oxygenation process.

### FIELD OF THE INVENTION

Preserving renal function during urologic surgery has been an elusive ambition for many years. The recognized technique of nephron sparing surgery has increased its application and practice in modern urology. The present invention relates to a novel method of perfusion using an oxygenated perfluorocarbon emulsion (PFC) via retrograde access to the kidney. The present invention also relates to delivery system to provide end organ oxygenation and even systematic oxygenation in the face of ischemic result.

### BACKGROUND OF THE INVENTION

The main limiting factor in nephron spanng surgery is the cross clamp time or ischemic threshold of the kidney. The susceptibility of the kidney to hypoxic insult is a result of the derailment of normal cellular metabolism. The cessation of aerobic respiration and oxidative phosphorylation results in anaerobic glycolysis which produces lactic acid and inorganic phosphates. These metabolic byproducts lower the intracellular pH and change the cytosolic milieu resulting in impaired cellular volume and solute regulation. Membrane polarity is lost, calcium influx occurs, lysosomes leak releasing catabolic enzymes which denature intra and extracellular matrix proteins, all of which culminates in cellular destruction and death. The cells most susceptible to hypoxic damage in the kidney are the proximal tubule cells located in the renal cortex.

Based on human and animal data it has been established that for open renal procedures no permanent organ damage occurs for a normothermic or warm ischemic interval of 30 minutes or less. If surface hypothermia is used to achieve cortical temperatures between 5° and 25° C. an additional three hours of renal protection during temporary ischemia is realized. Although this is easily applied to open renal surgery, surface cooling of the kidney presents several technical difficulties for the minimally invasive surgeon as well as increased operative time and expense. If the ischemic threshold can be increased by an endoscopic technique, this would allow both open and minimally invasive surgeons a novel method of in situ renal preservation in order to attempt more complex and challenging dissections in a safe and effective manner.

The feasibility of an endoscopic renal protective technique is dependent on the identification of an alternative oxygen delivery vehicle. The ideal oxygen carrier should be inexpensive, widely available, non-immunogenic, have favorable oxygen transpolt properties, present no infectious risk, and be without harmful side effects. Perfluorocarbons (PPC) are low molecular weight (450-550 Daltons) linear or cyclic hydrocarbon chains that dissolve gasses without covalent bonding. The hydrogen atoms from the carbon chain are replaced with fluorine or bromine atoms resulting in a chemically and biologically inert substance. The solubility of respiratory gasses depends solely on the amount of PFC available and the partial pressure of each gas, thus oxygen transport is based on Henry's linear law of partial pressures. Therefore, unlike hemoglobin, acidosis, alkalosis, 2,3-diphosphoglycerate, and temperature have little or no effect on oxygen (02) delivery. Eventually, PFC are processed by the reticuloendothelial system and then excreted as vapors from the lungs. However, because PFC is not soluble in water, it must be administered as emulsions. Particle size determines PFC stability, surface area available for gas transport, viscosity and half-life. Emulsions containing 45-60% PFC by weight/volume are ideally suited for oxygen transport. Oxygen{” (Alliance Pharmaceutical Corporation, San Diego, Calif.) was utilized in this experiment as the alternative 02 carrier due to its commercial availability, known properties, and approved FDA status (as a blood substitute). The present invention relates to process and device for renal oxygenation and protection during temporary ischemia via retrograde access through the urinary collecting system utilizing an oxygenated perfluorocarbon emulsion.

The purpose of the perfusion system described herein is to provide end organ oxygenation and even systemic oxygenation in the face of ischemic insult. The device(s) and delivery system described are intended to utilize the renal pelvis (urinary collecting system) and the biophysical phenomenon of pyelovenous and pyelosinus black flow. The novel urinary stent is deployed in a retrograde fashion though an intact bladder using current endoscopic techniques. The catheter is then externally connected to a delivery system that would deliver the perfusate directly to the renal collecting system while monitoring renal pressures and temperatures through the stent. The delivery system is able to fully oxygenate the perfusate (utilizing hollow core fibers), salvage used material, and regulate the delivery of the perfusate material in either a constant or pulsed pressure. The stent design may include safety measures to prevent inadvertent high renal collecting system pressures that could possibly result in a forniceal rupture.

These as well as other novel advantages, details, embodiments, features, and objects of the present invention will be apparent to those skilled in the art from the following detailed description of the invention, the accompanying drawings, which are useful in explaining the invention.

### BRIEF DESCRIPTION OF THE DRAWINGS

FIG. 1 depicts an embodiment of the stent of the present invention;

FIG. 2 depicts an embodiment of the stent of the present invention in cross-sectional view;

FIG. 3 depicts an embodiment of the stent of the present invention in cross-sectional view; and

FIG. 4 depicts an embodiment of the oxygenation delivery apparatus of the present invention.

### DETAILED DESCRIPTION OF THE INVENTION

For a better understanding of the present invention, reference may be had to the following detailed description taken in conjunction with the accompanying drawings.

Materials and Methods: Thirty mature female New Zealand White rabbits between 2.5 and 3.0 kg were randomized to one of five retrograde renal perfusion treatment groups: Group S=sham (no retrograde perfusion), Group NS=noml0themlic saline, Group CS=chilled saline, Group NPFC=normothermic PFC, and Group CPFC=chilled PFC. Regardless of the treatment group each animal underwent an identical surgical procedure as described below.

Prior to the initiation of the surgical procedure each animal was allowed liberal access to food and water and underwent baseline renal function determination, see Table 1. Ketamine (35-50 mg/kg) and Xylazine (5-10 mg/kg) were used intramuscularly for anesthesia induction followed by endotracheal intubation. An ear margin vein was cannulated and a 1 mL venous blood sample was removed for blood gas and creatinine analysis. Anesthesia was maintained throughout the procedure using inhaled isoflurane 2% with a tidal volume of 10-12 cc/kg and a respiratory rate of 25 breaths per minute. Intraoperative hydration was maintained with 0.9% normal saline. Each animal had its vital signs monitored and recorded throughout the duration of the procedure.

TABLE 1
Outcome Measures

| | | | Post | Post |
|---|---|---|---|---|
| | | Post- | Procedure | Procedure |
| | Pre-Operative | Procedure | Week 1 | Week 2 |
| Weight | X | X | X | X |
| Systemic | X | X | | |
| Blood Gas | | | | |
| Serum | X | | X | X |
| Creatinine | | | | |
| Urine Output | X | | X | X |
| and | | | | |
| Creatinine | | | | |
| Creatinine | X | | X | X |
| Clearance | | | | |
| Ischemic | | X | | |
| Interval | | | | |
| Retrograde | | X | | |
| Perfusion | | | | |
| Pressure | | | | |
| Renal Weight | | X | | X |
| Histologic | | X | | X |
| Score | | | | |

An 8-10 em midline laparotomy incision was made and the bladder was delivered into the operative field. The right renal hilum was identified and each structure: artery, vein, and ureter were carefully isolated. Caution was used to preserve the superior, inferior, lateral and posterior retroperitoneal attachments of the kidney to prevent postoperative vascular compromise when the quadruped animal was ambulatory. A 2 em midline cystotomy incision was made into the bladder identifying both urinary orifices. A 0.018″ 175 cm Essence Guidewire (Cordis Corporation, Johnson & Johnson, New Brunswick, N.J.) and 2.3 French (0.031″) 70 em Rapid Transit Catheter (Cordis Corporation, Johnson & Johnson, New Brunswick, N.J.) were used to gain retrograde access to the right renal pelvis. The extracorporeal portion of the ureteral catheter was secured to the abdominal wall. The end of the catheter was attached to a stopcock that was connected to a Hewlett Packard 78834A pressure monitor (Hewlett Packard, Palo Alto, Calif.) and a 40 mL syringe in the syringe pump (Harvard Apparatus, Inc., Holliston, Mass.). Then the right renal artery was occluded with an atraumatic pediatric bulldog clamp for 40 minutes. The retrograde perfusion rate was between 0.05 and 0.10 mL/min to maintain renal pelvic pressures between 50 and 65 mmHg, as determined in a previous experiment, in order to achieve pyelovenous backflow. The only variable was the choice and temperature of the retrograde perfusate. At the conclusion of the ischemic interval the bulldog clamp was removed and a 1 mL systemic venous blood sample was again taken for analysis. The untreated left kidney was then removed, wrapped in gauze and placed in iced normal saline for histopathologic processing as the control specimen for each animal. The retrograde ureteral catheter was removed and a 5 french pediatric feeding tube was passed ante grade from the bladder. The bladder was closed with a running 6-0 Monocryl suture, and then filled with 15 mL of normal saline to ensure that the closure was water tight. The wound was finally closed in three layers, and the animal allowed to recover.

Several different measures of renal function in this experiment were examined including serum creatinine, creatinine clearance, 24 hour urine output (using individual metabolic cages), and urinary creatinine concentration. Overall animal welfare was judged by weight and dietary intake. Each animal was assessed preoperatively, at surgery, and post-operatively at day 7 and 14. Renal function was determined by serum creatine and creatine clearance, while systemic venous blood gas parameters were measured immediately before and after the retrograde perfusion. Creatinine clearance (Cr Cl) as an estimation of the glomerular filtration rate (GFR) was used according to the following formula:

GFR=Cr Cl=(U×V/P×T)/W,

GFR=creatinine clearance per body weight (mL/min/kg), U=creatinine concentration in the urine (mg/dL), V=volume of urine excreted in 24 hours (mL), P=creatinine concentration in the serum (mg/dL), T=number of minutes in 24 hours (min), and W=the weight of the animal (kg)

At the end of a two-week survival period the animals were sacrificed and histopathologic examination and comparison was done. A single blinded pathologist (TJS), utilizing a novel ischemic grading scale (Table 2), graded one hundred random cortical fields per tissue specimen with a 40× lens. The cellular profile was evaluated for: tubular cell swelling, the loss of brush border, nuclear condensation, and nuclear loss or drop out. The scores potentially ranged from 0 to 300, with lower scores indicating preserved renal architecture.

TABLE 2
Renal Ischemic Grading Scale

| Score | Degree of Change |
|---|---|
| 0 | No abnormal features seen |
| 1 | Up to ⅓^{rd} of cells exhibit an altered profile |
| 2 | Between ⅓^{rd} and ⅔^{rds} of cells exhibit an altered profile |
| 3 | ⅔^{rds} or more of cells exhibit an altered profile |

The protocol was designed to detect a change in the endpoint between two histologic levels of ischemia equivalent to 1.7 standard deviations with approximately 80% power (alpha=0.05, 2-sided, two-sample t-test). Analysis of variance was used to assess treatment differences, testing for differences among sham, saline, and PFC perfusion cohorts, and between chilled and non-chilled perfusion cohorts. Additionally, each treatment group was compared individually to the sham (control) treatment group using the two-sample t-test. Pre and post-operative values were also compared using the paired t-test.

Results: Serum creatinine and GFR are commonly accepted indicators of overall renal function as presented in Table 3. Post-operatively all of the experimental groups experienced a rise in serum creatinine from baseline, which generally improved by the fourteen day after the ischemic insult. Several trends were apparent. The CPFC group had their serum creatinines return the closest to baseline, 0.68±0.14 mg/dL, at post operative day 14,0.85±0.10 mg/dL, while Group S had the largest increase in serum creatinine from baseline to post-operative day 14,0.80±0.10 and 1.10±0.52 mg/dL respectively. The final serum creatinine values (post-operative day 14) for Groups NS, CS, and NPFC were: 1.03±0.26, 1.07±0.28, and 1.32±0.55 mg/dL, respectively.

At post-operative day 7, the NPFC and NS groups had the least decrease in mean GFR (4.9±3.9 and 1.7±4.2 mL/min/kg), which was statistically significant (p<0.05), compared to the S, CS and CPFC groups (10.3±5.3, 9.4±7.0 and 5.8±3.0 mL/min/kg). At post-operative day 14, although not statistically significant, the NS, NPFC, and CPFC groups all had less decline in mean GFR compared to the S group: 2.3±3.3, 3.6±3.9, and 4.0±2.0 compared to 7.8 8.4 mL/min/kg respectively.

TABLE 3
Creatinine Clearance per Body Weight After Retrograde Renal
Perfusion (mL/min/kg)

| | | Post-Op | Post-Op | | |
|---|---|---|---|---|---|
| | Baseline | Day 7 | Day 14 | Week 1 Cr | Week 2 Cr |
| Cohort | CrCl | CrCl | CrCl | Difference | Difference |
| Group S | 15.1 | 4.8 | 7.3 | ⁻10.3 | ⁻7.8 |
| (Sham) | (9.9-20.7) | (2.8-6.6) | (2.4-14.0) | (⁻17.9-⁻3.3) | (⁻18.3-2.8) |
| Group NS | 9.5* | 7.8 | 7.2 | ⁻1.7* | ⁻2.3 |
| (Normothermic | (5.1-13.5) | (3.8-19.0) | (5.1-12.1) | (⁻7.0-5.5) | (⁻6.7-2.5) |
| Normal Saline) | | | | | |
| Group CS | 14.3 | 4.9 | 7.1 | ⁻9.4⁺ | ⁻7.2 |
| (Chilled Normal | (7.3-27.1) | (3.5-7.8) | (5.6-12.7) | (⁻20.9-⁻3.6) | (⁻21.5-0.0) |
| Saline) | | | | | |
| Group NPFC | 9.6* | 4.7 | 6.0 | ⁻4.9 | ⁻3.6 |
| (Normothermic | (5.8-13.3) | (2.4-7.8) | (2.6-8.4) | (⁻10.2-⁻1.7) | (⁻10.1-0.2) |
| Oxygent ™) | | | | | |
| Group CPFC | 12.0 | 6.2 | 8.0 | ⁻5.8⁺ | ⁻4.0 |
| (Chilled | (10.2-15.3) | (3.8-12.2) | (7.2-9.4) | (⁻10.4-⁻1.7) | (⁻6.1-⁻0.8) |
| Oxygent ™) | | | | | |

*Significantly different from sham procedures (p < 0.05)
⁺Chilled procedures significantly different from non-chilled procedures (p < 0.05)

There were no significant differences in the urine output during the 24 hour urine collection periods among the different treatment groups. Overall (not stratifying by treatment group), there was a significant decline between pre-operative baseline (median volume 141 mL) and post-operative day 7 (median volume 62.5 mL, p<O.OOOl) and day 14 (median volume 94.5 mL, p=O.OOl). All groups showed a decline in urine volume, following their procedure and unilateral nephrectomy.

The decrease in urinary concentration of creatinine for the 24 hour urine samples at post-operative day 7 and 14 was reduced for the NS, NPFC and CPFC groups (19.7±20.9, 25.5±49.5, 26.3±21.6 and 10.8±13.6, 23.5±40.8, 20.2±10.9 mg/specimen, respectively) compared to the S group (98.3±67.1 and 89.1±76.6 mg/specimen). This was statistically significant (p<0.05), indicating that these groups had less of a decrease in the amount of filtered (and excreted) creatinine at both post operative day 7 and 14 compared to the sham group (Table 4).

TABLE 4
24 Hour Urine Creatinine Concentration Before and After
Retrograde Renal Perfusion (mg/specimen)

| | | | Post-Op | Week 1 | Week 2 |
|---|---|---|---|---|---|
| | Baseline | Post-Op Day 7 | Day 14 | Urine Cr | Urine Cr |
| Cohort | Urine Cr | Urine Cr | Urine Cr | difference | Difference |
| Group S | 175.8 | 77.5 | 86.7 | ⁻98.3 | ⁻89.1 |
| (Sham) | (104-268) | (70-89) | (59-109) | (⁻204-⁻20) | (⁻205-⁻3) |
| Group NS | 110.0 | 90.3 | 99.2 | ⁻19.7* | ⁻10.8* |
| (Normothermic | (95-135) | (44-114) | (86-106) | (⁻51-1) | (⁻29-4) |
| Normal Saline) | | | | | |
| Group CS | 139.8 | 91.0 | 99.3 | ⁻48.8 | ⁻40.5 |
| (Chilled Normal | (93-234) | (43-135) | (50-116) | (⁻136-⁻13) | (⁻121-11) |
| Saline) | | | | | |
| Group NPFC | 120.8 | 95.3 | 97.3 | ⁻25.5* | ⁻23.5* |
| (Normothermic | (90-192) | (84-114) | (74-111) | (⁻116-24) | (⁻92-21) |
| Oxygent ™) | | | | | |
| Group CPFC | 118.0 | 91.7 | 97.8 | ⁻26.3* | ⁻20.2* |
| (Chilled | (104-268) | (81-105) | (72-120) | (⁻55-9) | (⁻34-⁻4) |
| Oxygent ™) | | | | | |

*Significantly different from sham procedure (p < 0.05)

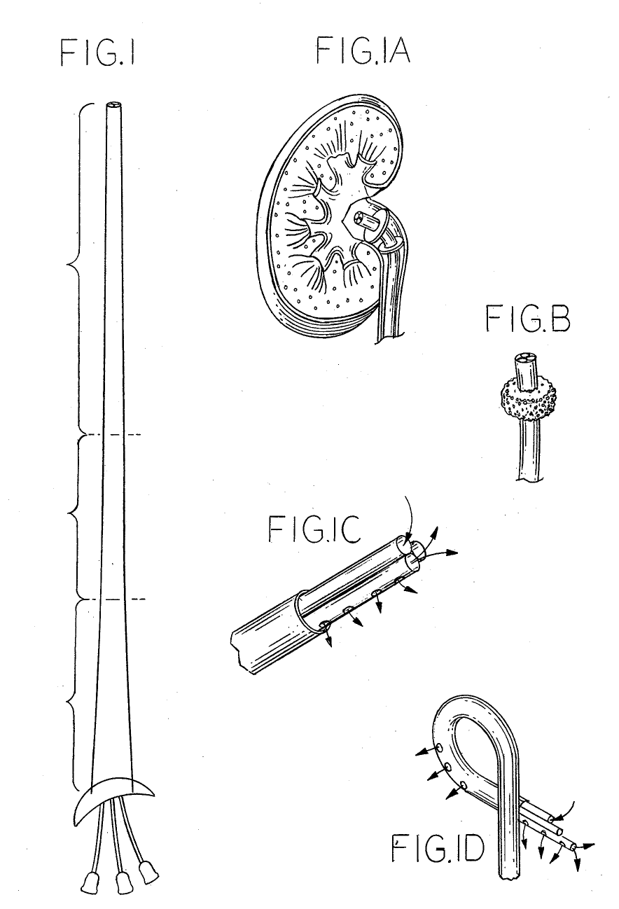

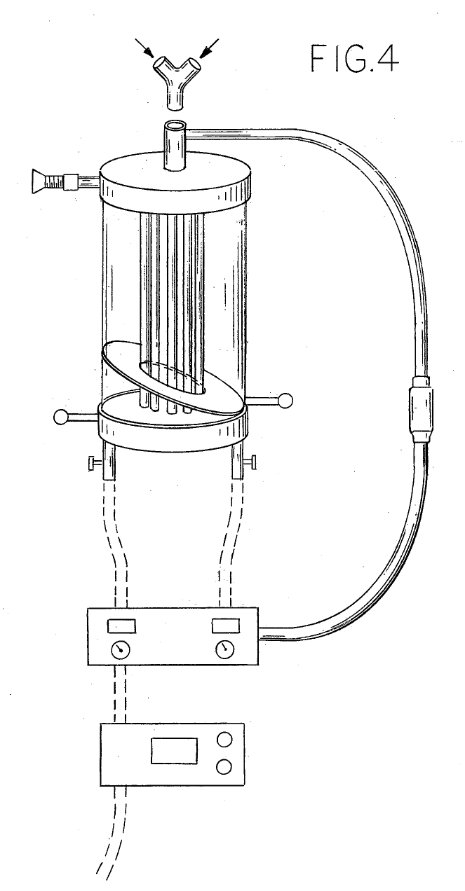

Immediately before and after the retrograde renal perfusion the systemic venous partial pressure of oxygen, p02, was measured. The post procedure systemic venous p02's were statistically higher in the NPFC and CPFC groups (75.33±14.90 and 69.83±13.30 mmHg) than those of the S group, 59.83±19.91 mmHg. These systemic p02 levels were elevated higher in the PFC groups than in any other treatment group (Group NS=73.83±10.93 and Group CS=62.17±9.40 mmHg), providing evidence that the retrograde renal perfusion and oxygen delivery was successful. This was visually confirmed as the normally dark venous blood of the renal vein turned arterial red during the course of the retrograde renal perfusion (FIGS. 4 and 5). The NPFC group had the most improvement in their systemic oxygenation parameters (an increase of 26.33 mmHg) compared to the minor improvement noted in the other groups (NS, CS, CPFC, and Shad 9.33, 9.17, 10.00, and 0.17 mmHg increases respectively) as demonstrated in FIG. 1. The systemic venous partial pressure of carbon dioxide, pCO_{z}, and pH results did not express any significant alteration is the acid base axis.

The individual animal's weight was used as an indicator of overall well being and health. Overall the animal weights did decrease significantly from baseline (mean 2.64 kg) to post operative day 7 (mean 2.32 kg, p<O.OOI) and day 14 (mean 2.49 kg, p=0.004). Only the CS and CPFC (chilled groups) had statically significant weight declines at post operative day 7. However, these groups regained enough body weight by post operative day 14 that this was no longer statistically different than preoperative values.

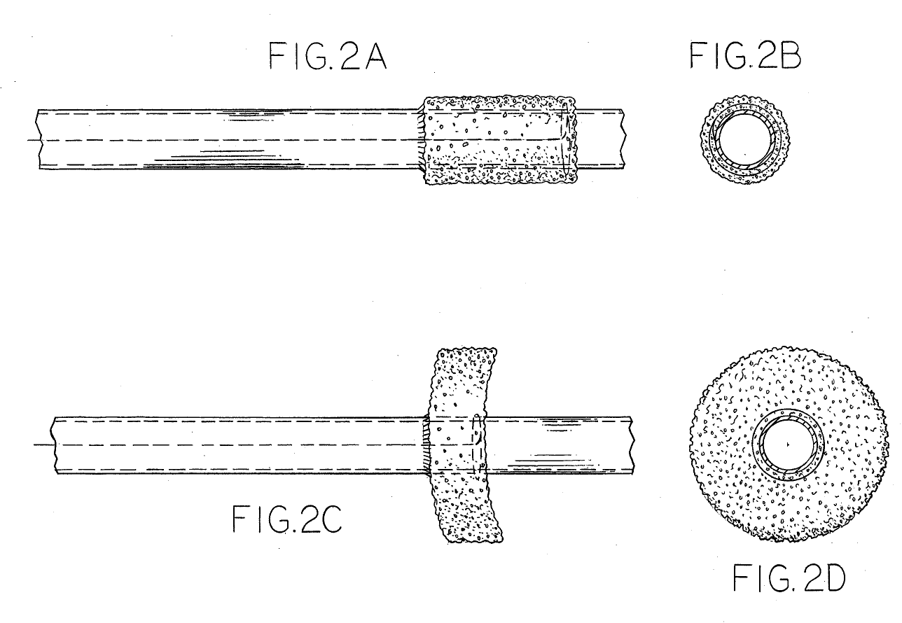

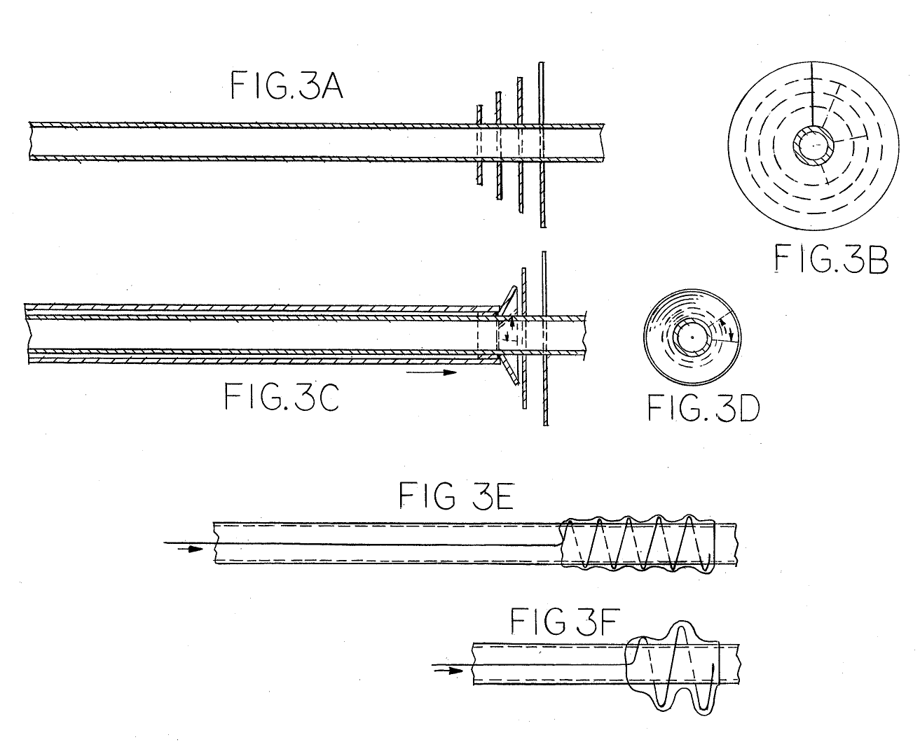

Blinded histopathologic examination revealed that each of the retrograde perfusion groups had less injury demonstrated from the ischemic insult (a lower histologic score) than the sham group, Table 5. The mean histologic scores of the groups were: control (no ischcmia or retrograde perfusion, nephrectomy at time of surgery) 5.5±2.3, Group S (ischemia but no retrograde perfusion) 33.3±16.8, Group NS 22.7±15.9, Group CS 12.3±9.5, Group NPFC 13.0±13.5, and Group CPFC 8.7±4.5. The chilled PFC versus the sham (p=0.003), chilled saline versus sham (p=0.009), and normothermic PFC versus sham (p=O.OII) all demonstrated statistically significant protective histologic findings. The microscopic findings of the normothermic PFC versus the sham cohort is illustrated in FIGS. 2 and 3, respectively.

TABLE 5
Blinded histopathologic ischemic scores of the experimental groups

| | | | Difference | |
|---|---|---|---|---|
| | Mean | Standard | from | |
| Cohort | score | deviation | control | P values |
| Control | 5.5 | 2.3 | — | — |
| | (3-9) | | | |
| Group S | 33.3 | 16.8 | 27.8 | — |
| (Sham) | (11-57) | | | |
| Group NS | 22.7 | 15.9 | 17.2 | P = 0.164 |
| (Normothermic Normal | (7-51) | | | |
| Saline) | | | | |
| Group CS | 12.3* | 9.5 | 6.8 | p = 0.009 |
| (Chilled Normal Saline) | (2-30) | | | |
| Group NPFC | 13.0* | 13.5 | 7.5 | p = 0.011 |
| (Normothermic | (0-38) | | | |
| Oxygent ™) | | | | |
| Group CPFC | 8.7* | 4.5 | 3.2 | p = 0.003 |
| (Chilled Oxygent ™) | (2-14) | | | |

*Significantly different from sham procedures

Discussion: In this feasibility study retrograde infusion of a novel oxygen carrier, PFC, through the renal collecting system resulted in successful systemic and renal oxygenation. FUlihem10re, pathologic and biochemical indices demonstrated renal preservation and improved renal function in these groups compared to the sham animals.

The rabbit model for this pilot study was chosen based on published data regarding perfusion pressures, characterized responses to ischemic injury, and previously reported experience utilizing PFC in this particular animal. In spite of the structural and functional differences between human and rabbit kidneys these data demonstrate the feasibility and merit of retrograde renal and systemic oxygen delivery. The rabbit has a single papillary renal unit compared to the compound urinary collecting systems seen in larger animals and humans. Fluid dynamic analysis and distribution mapping would become necessary in a compound urinary collecting system model in order to fully extrapolate these results. Also with the rabbit model, size and instrumentation were scaled down possibly confounding the results.

The animals in each experimental arm tolerated the procedure well without any observed complications to the retrograde renal perfusion. No renal pelvic ruptures, urinomas, infections, ureteral strictures or premature deaths occurred. No adverse effects due to the use of PFC were encountered. If any embolic phenomenon occurred it was subclinical and did not result in any morbidity for the experimental groups.

Oxygenating the kidney via the urinary collecting system provided a renal protective effect. The improved systemic venous p02's in the saline and PFC cohorts suggests that the transportation and unloading of oxygen through the urinary collecting system was successful in providing systemic oxygenation in addition to renal oxygenation. The sham animals, as one would expect, had no increase in systemic oxygenation while the normothermic PFC cohort had the largest increase in systemic p02. It is possible that due to the higher level of molecular oxygen concentration and saturation in the PFC emulsion compared to the saline solution, that renal tissue was more susceptible to reperfusion injury. The increased amount of O₂ delivered in the PFC groups would allow the generation of more free radical species thus temporizing and limiting the beneficial effect of the more oxygen rich PFC. Additionally the chilled retrograde perfusion groups did not realize the renal protective effects demonstrated in other experiments. This could be attributed to the slow rate of material delivery, thermodynamic conductive effect of the ureteral catheter, imprecise temperature control, or the systemic heat sink of the retroperitoneal tissue. Without intrarenal temperature monitoring this was difficult to control for and as such an inherent limitation of this study.

To date the most effective and popular method used to preserve renal function for prolonged ischemic intervals is surface hypothermia by cooling the kidney with iced saline slush. Hypothermia decreases metabolic activity and 02 consumption to 5% of normal when the cortical temperature reaches lSoC. Ward et al. classified the ideal renal protective temperature as ISoC, but its application to in situ practice has proven difficult secondary to the influence from adjacent organs, ambient temperatures, and inhomogeneous cooling of the tissue, and the potential for permanent hypothermic injury. Other approaches to renal cooling use heat exchange coils and continuous or intermittent arterial perfusion with cold saline solutions. Landman and colleagues recently described the endoscopic transureteral circulation of ice cold saline] to achieve renal hypothermia. Landman, et al used 0.9% normal saline at −1.7° C. circulating at 85 ml/min. The 3 L bags were 60 cm above the level of the kidney as higher pressures induced pyelotubular backflow. Landman and colleagues concluded that the renal tissue was preserved as well as surface cooled kidneys, but surface cooling was slightly more efficient at lowering renal cortical temperatures. Despite the protective effect of surface hypothermia, the rate of post-operative renal failure after open partial nephrectomy in humans can still approach 14%. The differences in results, clinical outcomes, and difficulty in adapting these methods has resulted in a search for innovative techniques of renal preservation.

Intravascular perfusion of the kidney using PFC was first demonstrated by Beisang et al, in 1970. Nakaya and colleagues also intravascularly perfused rabbit kidneys at room temperature for 9 hours with a PFC emulsion. They determined that the renal metabolic parameters were improved compared to electrolyte perfused kidneys. Brasile et at, described warm (32° C.) ex vivo renal preservation in canine kidneys that were then successfully autotransplanted after 6 hours of intravascular PFC perfusion. To our knowledge no one has attempted renal or systemic oxygenation through the collecting system utilizing a retrograde approach.

Pyelorenal backfiow is the condition where the contents of the renal pelvis and calyceal system penetrate the peripelvic sinus tissue (pyelosinus backflow), the renal vein (pyelovenous backflow), or the collecting ducts, tubules, and renal interstitium (intrarenal backflow). Thomsen et al carried out a series of experiments on rabbits to determine pyelorenal backflow pressures in normal and ischemic kidneys. They demonstrated that intrarenal backflow occurred at lower renal pelvic pressures as renal artery occlusion time increased. They also found that the increased susceptibility to intrarenal backfiow was reversible for ischemic times of 40 min or less in the acute setting. During arterial occlusion intrarenal backflow occurred at pressures between 58-77 mmHg (average 60 mmHg). Subcapsular extravasation was encountered at pressures of 79-116 mmHg, and was accompanied by a quick decrease in renal pelvic pressure. It was our concept to take advantage of this phenomenon to oxygenate the kidney during times of ischemia.

The data presented here (global renal function, serum creatinine, and creatinine clearance) suppOli the feasibility of this retrograde oxygenation technique. Postoperatively, the retrograde perfused cohorts did statistically better than the sham cohort with respect to creatinine clearance. This benefit was more pronounced for the nom10them1ic groups than the chilled groups, but the statistical significance did disappear after two weeks. Renal function was better or at least preserved in all the groups compared to the sham cohort. The results could be influenced by the fact that the pre-operative baseline values were based on twice the functional renal mass as the post-operative values because of the contralateral nephrectomy at the time of surgery. However, in order to establish the safety of the retrograde perfusion and to make the animal dependent on that particular renal unit this approach was necessary.

The delivery system of the present invention that provides end organ oxygenation and even systematic oxygenation contains a retrograde oxygenation and perfusion stent (ROPS) and an oxygentation delivery apparatus (ODA).

The Retrograde Oxygenation and Perfusion Stent (ROPS): The stent is preferably constructed of either silicone, polyurethane, or possibly coated with an inert hydrophobic coating that would not interact with the perfusate material. The stent is preferably constructed of material that is flexible with a low surface coefficient of friction to allow sterile retrograde placement over a guidewire or through a sheath device. The stent of the present invention should employ at least two and possibly more channels to allow flow of the perfusate from the device to the renal pelvis then to a back out to a collection apparatus. The stent may also include various vital sign monitors, such as a renal pressure monitor, temperature monitor, and even an oxygenation monitor. The outside diameter of the stent is preferably in the range from 6 to 14 french to allow easy placement with currently accepted endourologic equipment. The inner channels can range from 2 to 12 french depending on the viscosity and temperature of the perfusate material. The perfusate may be a perfluorocarbon emulsion, Oxygent® (Alliance Pharmaceutical Corp), delivered through a 2.3 french catheter. The length of the stent may be variable to allow manipulation outside the intact urinary system, approximately 40-60 em, with a single standard length stent available for both men and women.

The stent is capable of being anchored into the renal pelvis in a temporary way during the retrograde oxygenation process. The stent also is able to increase the resistance of the ureteropelvic junction (UPJ) in order to create perfusion pressures adequate to induce pyclosinus and pyelovenous backflow. The anchoring device may be a sponge type material with controlled pore size to allow distal delivery of the perfusate and then outflow of the material down the ureter or back into the stent into a collection apparatus (controlled by low grade suction). Alternatively, the anchoring device could be of a cone, inverted cone, or series of flexible discs that would seal off the UPJ while employing safety pores that would open under defined pressure thresholds. Additionally, the anchoring device could be a curl in the stent, change in stent diameter, or inflation balloon to anchor the stent in the correct position. The stent may employ radiopaque markers that will be easily identifiable on fluoroscopic exam to ensure proper device placement. The anchoring device is preferably retractable (though a sheath) or flexible enough to allow removal without inducing an injury.

The Oxygenation Delivery Apparatus (ODA): This ODA contains at least one reservoir that is capable of circulating a perfusate though a hollow fiber oxygenation system while allowing the temperature of the perfusate to be modified through heat exchange coils or cooling coils. The ODA is contains an external source of oxygen or potentially other gas. The ODA may measure the end oxygenation level of the perfusate though a draw out port or may employ an integrated laser pulse oxygenation sensor. Once the material is oxygenated, the ODA is capable of diverting the material to a holding chamber that will maintain the temperature and oxygenation of the perfusate until time of renal delivery. The hollow fiber oxygenation component may be removable and replaceable. After the holding chamber the material would transfers through a delivery pump that may relay the perfusate to the renal collecting system in a pulsed, constant or variable manor. The pressure is preferably controllable. The ODA may also contain a separate collection apparatus that may allow collection of the used material for recycling through the oxygenation chamber. The ODA preferably is easily connect and is compatible with the stent device. The ODA is preferably small, portable and reusable.

In the foregoing specification, the present invention has been described with reference to specific exemplary embodiments thereof. It will be apparent to those skilled in the art, that a person understanding this invention may conceive of changes or other embodiments or variations, which utilize the principles of this invention without departing from the broader spirit and scope of the invention. The specification and drawings are, therefore, to be regarded in an illustrative rather than restrictive sense.

1. A delivery system to provide systematic oxygenation during retrograde perfusion comprising:
a stent having at least two interior channels in the range of 2 to 12 french to allow flow of perfusate to an organ, the stent having an outside diameter in the range of 6-14 french
at least one physiological detector coupled to the stent for monitoring vital signs of a patient;
a retractable anchoring device coupled to the stent for anchoring the stent in an organ;
an oxygenation delivery device connectable to the stent capable of circulating perfusate to the stent.

2. The delivery system in claim 1 wherein the stent includes an inert bydrophobic coating.

3. The delivery system of claim 1 wherein in the anchoring device is comprised of a sponge material with controlled pore sizes allowing delivery of the perfusate.

4. The delivery of the system of claim 1 wherein the anchoring device is comprised of at least two flexible discs.
